(19) United States
(12) Patent Application Publication
(10) Pub. No.: US 2025/0268461 A1
(43) Pub. Date: Aug. 28, 2025

(54) INSTRUMENT SHAPE ESTIMATION

(71) Applicant: AURIS HEALTH, INC., Santa Clara, CA (US)

(72) Inventors: Namita Anil KUMAR, Redwood City, CA (US); Daniel Shalom ELIAHU, Alamo, CA (US); Chauncey F. GRAETZEL, Palo Alto, CA (US); William Lanphier CHAPIN, Redwood City, CA (US); Mina CHOW, Redwood City, CA (US); Qasim MAHMOOD, Redwood City, CA (US); Yuriy MALININ, Palo Alto, CA (US)

(21) Appl. No.: 18/586,334

(22) Filed: Feb. 23, 2024

(51) Int. Cl. A61B 1/005; A61B 34/10; A61B 34/20

(52) U.S. Cl. CPC A61B 1/009; A61B 34/10; A61B 34/20; A61B 2034/104; A61B 2034/2051

(57) ABSTRACT

A system includes an elongate instrument, such as an endoscope, having a plurality of position sensors associated therewith, the plurality of position sensors being distributed along a length of the instrument. The system further includes control circuitry configured to determine positions and orientations of each of the plurality of position sensors, determine a curve that passes through each of the respective positions of the plurality of position sensors and the respective orientations, and estimate a shape of the elongate instrument based on the curve.

### BACKGROUND

The present disclosure relates to the field of medical procedures. Various medical procedures involve the use of instruments comprising an elongate flexible shaft, which can be advanced at least partially within the body of a subject. The shape of such instruments within the body can impact how the instrument interacts with the anatomy and how a user may wish to advance and/or articulate the instrument.

### SUMMARY

Described herein are systems, devices, and methods to facilitate the estimation of the shape of an elongate instrument positioned at least partially within a subject. Such shape estimation can be facilitated by implementation of a plurality of sensors, such as electromagnetic (EM) sensors, along a length of an elongate member, which may be the instrument itself, or may be secondary instrument/component that runs along a length of the instrument, such as within a working channel thereof. Position and orientation information relating to the sensors can be utilized to determine a spline/curve that runs through the determined sensor positions at the determined sensor orientations. Such curve may provide an estimate of the shape of the instrument, at least with respect to the length of the instrument spanned by the sensors. Matching of the spline/curve to the sensor positions/orientations may be based at least in part on the determination/identification of a curve that indicates sensor-to-sensor length(s) that most closely match known physical sensor-to-sensor lengths of the sensor-integrated member. The slope of the determined shape-matching curve at positions corresponding to the measured sensors can be based on quaternion values associated with the respective sensor readings, from which curve slope derivatives can be derived. Once the instrument shape has been determined, a representation of the tracked instrument can be rendered on a graphical interface that represents the shaped instrument in a proper position and shape with respect to a virtual model of the anatomy in which the instrument is disposed.

In some implementations, the present disclosure relates to a system comprising an elongate instrument having a plurality of position sensors associated therewith, the plurality of position sensors being distributed along a length of the elongate instrument, and certain control circuitry. The control circuitry is configured to determine positions and orientations of each of the plurality of position sensors, determine a curve that passes through each of the respective positions of the plurality of position sensors and the respective orientations, and estimate a shape of the elongate instrument based on the curve.

The plurality of position sensors can be integrated with a shaft of the elongate instrument. In some examples, the plurality of position sensors are integrated with an elongate member disposed at least partially within a working channel of the elongate instrument.

The control circuitry can be further configured to render a representation of at least a portion of the elongate instrument on a graphical interface, wherein the representation of the at least a portion of the elongate instrument has a shape that is based on the estimated shape of the elongate instrument. In some implementations, the curve is a third-degree Hermite spline.

In some embodiments, the control circuitry is further configured to determine sensor link lengths associated with the plurality of position sensors. For example, the curve can be based at least in part on the sensor link lengths. At least one of the sensor link lengths may span between non-adjacent sensors of the plurality of position sensors. In some examples, the control circuitry is further configured to re-configure the sensor link lengths during operation of the elongate instrument.

In some implementations, the present disclosure relates to a system comprising control circuitry configured to communicatively couple to an elongate instrument having a plurality of position sensors distributed along a length thereof. The control circuitry is configured to determine relative positions of the plurality of position sensors with respect to the length of the elongate instrument, determine sensor link lengths associated with the plurality of position sensors, the sensor link lengths spanning lengths of the elongate instrument between pairs of the plurality of position sensors, and generate a curve that represents a shape of the elongate instrument based on the sensor link lengths.

Determining the relative positions of the plurality of position sensors and determining the sensor link lengths can involve accessing pre-determined parameters associated with the elongate instrument.

In some implementations, the control circuitry is further configured to determine current positions of the plurality of position sensors relative to a coordinate system and determine current orientations of the plurality of position sensors relative to the coordinate system, wherein the curve is based on the current positions and the current orientations. Further, the control circuitry can be further configured to generate one or more curve segments based on one or more of the current orientations and compare lengths of the one or more curve segments to respective ones of the sensor link lengths, wherein the curve is based on the comparing. Further, the control circuitry can be further configured to determine a total length of the curve, compare the total length of the curve to a predetermined length of the elongate instrument, and determine that the curve is a best-fit curve based on the comparing the lengths of the one or more curve segments and the comparing the total length of the curve.

The elongate instrument can be an elongate probe configured to be disposed within a working channel of an endoscope. In some embodiments, determining the sensor link lengths is performed in real-time in connection with the determining the curve.

In some implementations, the present disclosure relates to a method comprising determining positions of a plurality of position sensors distributed along a length of an elongate instrument relative to a coordinate system, determining orientations of the plurality of position sensors relative to the coordinate system, and generating a curve that passes through each of the positions of the plurality of position sensors at the respective orientations of the plurality of position sensors.

The method can further comprise inserting an elongate probe within a working channel of the elongate instrument, the plurality of position sensors being integrated with the elongate probe. The method can further comprise generating graphical interface data representing an anatomical model and a representation of a segment of the elongate instrument conforming to a shape that matches the curve within the anatomical model. The method can further comprise determining sensor link segments associated with the plurality of position sensors, wherein the curve is based on lengths of the sensor link segments.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates a robotic surgical system in accordance with one or more embodiments.

FIG. 2 illustrates medical system components that may be implemented in a robotic surgical system in accordance with one or more embodiments.

FIG. 3 is a block diagram illustrating a robotic instrument shape/position tracking system in accordance with one or more embodiments.

FIG. 4 shows a robotically controllable instrument assembly in accordance with one or more embodiments.

FIG. 5 illustrates a medical instrument having an elongate shaft associated with a plurality of sensors in accordance with one or more embodiments.

FIG. 6 illustrates a medical instrument having an elongate shaft associated with a plurality of sensors in accordance with one or more embodiments.

FIG. 7 illustrates a user interface for displaying a shape of an instrument and related information in accordance with one or more embodiments.

FIG. 8 is a flowchart illustrating a process for shape estimation of an instrument in accordance with one or more embodiments.

FIG. 9 is a flowchart illustrating a process for fitting a curve to position sensors associated with an instrument in accordance with one or more embodiments.

### DETAILED DESCRIPTION

Although certain preferred embodiments and examples are disclosed below, the inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Robotic Surgical Systems

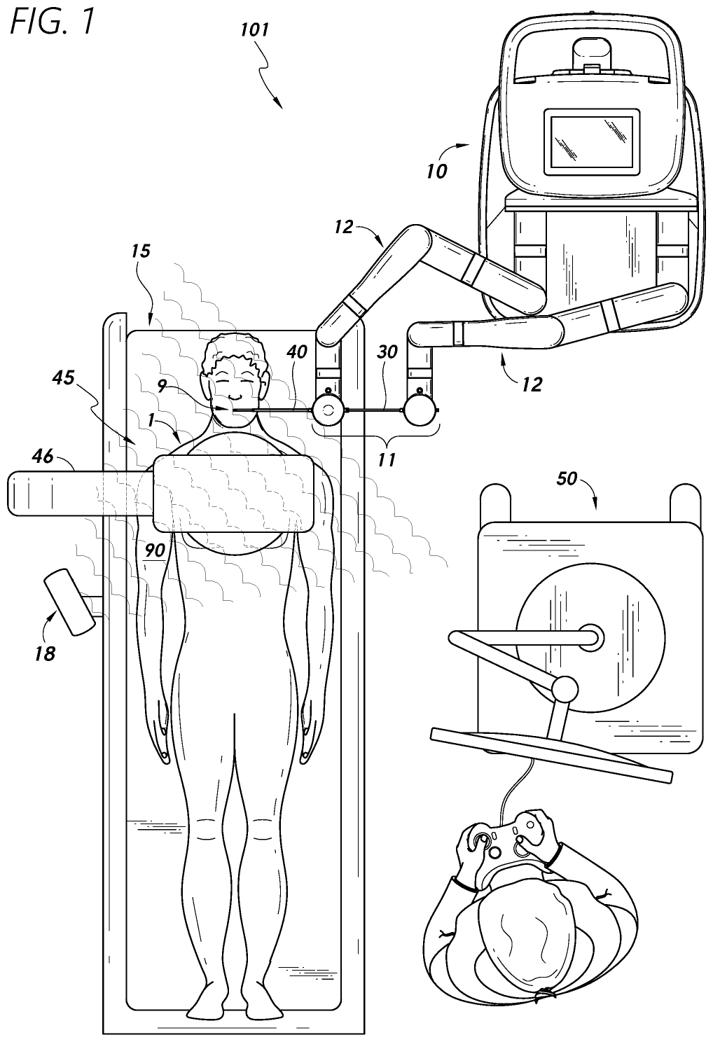

FIG. 1 illustrates a medical/surgical system 101 in accordance with one or more embodiments. For example, the system 101 can be arranged for diagnostic and/or therapeutic bronchoscopy, as shown. The system 101 may include and utilize a robotic system 10, which may be implemented as a robotic cart, for example. Although the system 101 is shown as a cart-based system, it should be understood that concepts disclosed herein may be implemented in any type of robotic system/arrangement, such as robotic systems employing rail-based and/or table-based robotic end-effectors/manipulators. The robotic system 10 may comprise one or more robotic arms or other robotic positioner(s) 12 configured to position a medical instrument, such as a steerable endoscope or other elongate instrument 30. For example, the elongate instrument 30 may be advanced through a natural orifice access point (e.g., the mouth 9 of the patient 1, positioned on a table 15 in the present example) to deliver diagnostic and/or therapeutic treatment. Although described in the context of a bronchoscopy procedure, it should be understood that the robotic system 10 can be implemented for other types of procedures, such as gastro-intestinal (GI) procedures involving a gastroscope or other specialized endoscope.

With the robotic system 10 properly positioned, the robotic positioner(s) 12 and/or instrument driver(s) 11 thereof may insert the steerable instrument 30 into the patient 1 robotically, manually, or a combination thereof. In some implementations, the instrument 30 may be advanced within an outer sheath 40, which may be coupled to, and/or controlled by, a robotic positioner/manipulator in some implementations. For example, the instrument 30 and the sheath 40 may be each coupled to a separate instrument driver from the set of instrument drivers 11. The instrument drivers 11 can be repositionable in space by manipulating the one or more robotic positioners 12 into different angles and/or positions.

The elongate instrument 30 may be directed down the patient's trachea and lungs after insertion and advanced to a target destination or operative site. In order to enhance navigation through the patient's lung network and/or reach the desired target, the instrument 30 may be manipulated to telescopically extend from the outer sheath 40 to obtain enhanced articulation and greater bend radius. The use of separate instrument drivers 11 allows the instrument 30 and sheath 40 to be driven independently of each other.

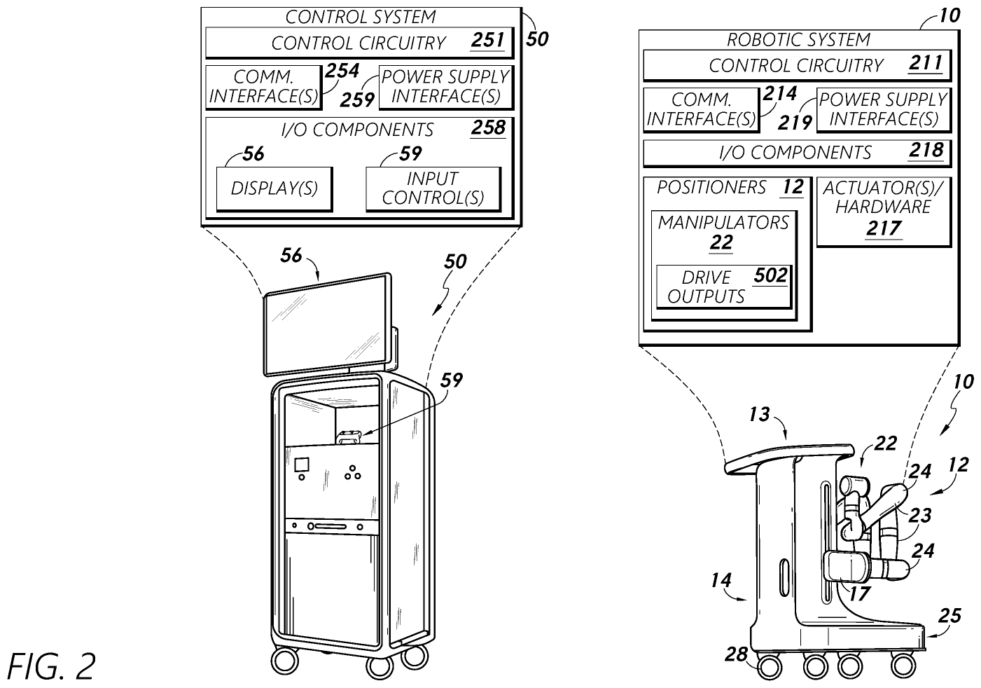

The system 101 may also include a control system 50 (e.g., mobile tower), described in detail below with respect to FIG. 2. The control system 50 may be communicatively coupled (e.g., via wired and/or wireless connection(s)) to the robotic system 10 to provide support for controls, electronics, fluidics, optics, sensors, and/or power to the robotic system 10. Placing such functionality in the control system 50 can allow for a smaller form factor robotic system 10 that may be more easily adjusted and/or re-positioned by operating staff. Additionally, the division of functionality between the robotic system 10 and the control system 50 can reduce operating room clutter and facilitate efficient clinical workflow.

The various components of the system 101 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. In some embodiments, the various communication interfaces can include wireless technology such as Bluetooth, Wi-Fi, near-field communication (NFC), or the like. Furthermore, in some embodiments, the various components of the system 101 can be connected for data communication, fluid exchange, power exchange, and so on via one or more support cables, tubes, or the like.

The system 101 includes an electromagnetic (EM) field generator 18, which is configured to broadcast an EM field 90 that is detected by EM sensors associated with the instrument 30, such as integrated with an elongate shaft of the instrument 30 along a length thereof or along a length of an elongate member disposed at least partially within a working channel of the instrument 30. In some implementations, inductive EM sensing is implemented, in which the electromagnetic field 90 induces small currents in coils of the EM position sensors, which may be analyzed to determine a position and angle/orientation of the EM sensors relative to the EM field generator 18. Other types of EM sensing may be implemented, such as sensing using magneto-resistance and/or Hall-effect sensors. The instrument 30 (e.g., endoscope) can include other types of sensors, such as a shape sensing fiber, accelerometer(s), gyroscope(s), satellite-based positioning sensor(s) (e.g., global positioning system (GPS) sensors), radio-frequency transceiver(s), and so on, which may be used in addition to, or as an alternative to, EM sensors. In some embodiments, a sensor on a medical instrument can provide sensor data to a control circuitry of the system 101, which is then used to determine a position, orientation, and/or shape of the instrument 30. Although EM fields and EM sensors are described as examples herein, it should be understood that position sensing systems and position sensors disclosed herein may be any type of position sensing systems and sensors known in the art, such as optical or image-based position sensing systems/sensors, ultra-sonic sensing, or shape sensing fiber position sensing systems/sensors, such as Optical Shape Sensing (OSS) or Fiber Bragg Grating (FBG) sensing systems/sensors.

The system 101 further comprises an imaging device 45, such as a fluoroscopic system configured to provide real-time images (e.g., X-ray) of the internal anatomy of the patient 1 and the instrument 30. The imaging device 45 can comprise an X-ray source and detector mounted on a C-shaped arm support 46, allowing for flexibility in positioning around the patient 1 to capture images from various angles without moving the patient 1. Use of the imaging device 45 can provide visualization of internal structures to guide the robotic insertion and navigation of the instrument 30. Use of the imaging device 45 can enhance the efficacy and safety of a medical procedure, such as a bronchoscopy, by providing clear, continuous visual feedback to the operator. In some implementations, the imaging device 45 is integrated with the one or more other components of the system 101. For example, fluoroscopy images generated using the imaging device 45 can be utilized by control circuitry of the system 101 in generating instrument navigation control signals to improve navigation accuracy. In some implementations, the shape of the instrument 30 can be segmented from the a fluoroscopic image and used to refine airway segmentation or any sensor-based shape estimate.

FIG. 2 illustrates medical system components that may be implemented in a robotic surgical system, such as the system 101 of FIG. 1. FIG. 2 shows example embodiments of a control system 50 and a robotic system 10, which can be implemented as a tower and a robotic cart, respectively. The control system 50 can be coupled to the robotic system 10 and operate in cooperation therewith to perform a medical procedure on a patient. For example, the control system 50 can include communication interface(s) 254 for communicating with communication interface(s) 214 of the robotic system 10 via a wireless or wired connection (e.g., to control the robotic system 10). Further, in some embodiments, the control system 50 can communicate with the robotic system 10 to receive position data therefrom relating to the position of sensors integrated with an instrument/member controlled by the robotic system 10. In some embodiments, the control system 50 can communicate with an EM field generator to control generation of an EM field in an area around a patient. The control system 50 can further include one or more power supply interface(s) 259.

The robotic system 10 can be arranged in a variety of ways depending on the procedure. The robotic system 10 can include one or more robotic positioners 12 (e.g., arms) configured to engage with and/or control, for example, an endoscope to perform one or more aspects of a procedure. As shown, each robotic positioner 12 can include multiple segments 23 coupled to joints 24, which can provide multiple degrees of movement/freedom. The robotic system 10 may be configured to receive control signals from the control system 50 to perform certain operations, such as to position one or more of the robotic positioners 12 in a particular manner, manipulate an instrument, and so on. In response, the robotic system 10 can control, using certain control circuitry 211 thereof, actuators 217 and/or other components of the robotic system 10 to perform the operations. For example, the control circuitry 211 may control insertion, articulation, and/or roll of a shaft of an endoscope or other elongate instrument by actuating drive output(s) 502 of the manipulator(s) 22 (e.g., end-effectors) coupled to a base of a robotically-controllable instrument. The robotic system 10 can include one or more power supply interfaces 219.

The robotic system 10 can include a support column 14, a base 25, and/or a console 13. The console 13 can provide certain user I/O components 218, such as a user interface for receiving user input and a display screen (or a dual-purpose device such as, for example, a touchscreen) to provide the physician/user with both pre-operative and intra-operative data. The column 14 may include a positioner support 17 (also referred to as a “carriage”) for supporting the deployment of the one or more robotic positioners 12. The positioner support 17 may be configured to vertically translate along the column 14. Vertical translation of the positioner support 17 allows the robotic system 10 to adjust the reach of the robotic positioners 12 to meet a variety of table heights, patient sizes, and physician preferences. The base 25 can include wheel-shaped casters 28 that allow for the robotic system 10 to easily move around the operating room prior to a procedure. After reaching the appropriate position, the casters 28 may be immobilized using wheel locks to hold the robotic system 10 in place during the procedure.

The joints 24 of the positioner(s) 12 can each be independently-controllable and provide an independent degree of freedom available for instrument navigation. In some embodiments, each of the positioners 12 has seven joints, and thus provides seven degrees of freedom, including “redundant” degrees of freedom. Redundant degrees of freedom allow the robotic positioners 12 to position their respective manipulators (e.g., drivers) 22 at a specific position, orientation, and trajectory in space using different linkage positions and joint angles. This allows for the robotic system 10 to position and direct a medical instrument from a desired point in space while allowing the physician to move the joints 24 into a clinically advantageous position away from the patient to create greater access, while avoiding collisions.

The manipulator 22 may be couplable to an instrument base/handle, which may be attached using a sterile adapter component in some instances. The combination of the manipulator and coupled instrument base, as well as any intervening mechanics or couplings (e.g., sterile adapter), can be referred to as a manipulator assembly 11, or simply a manipulator. Manipulator assemblies can provide power and control interfaces. For example, the interfaces can include connectors to transfer pneumatic pressure, electrical power, electrical signals, and/or optical signals from the robotic positioner 12 to the coupled instrument base. Manipulator assemblies may be configured to manipulate medical instruments (e.g., surgical tools/instruments) using techniques including, for example, direct drives, harmonic drives, geared drives, belts and pulleys, magnetic drives, and the like.

The control system 50 may include control circuitry 251 configured to cause the components of the robotic surgical system to actuate/control any of the various system components, including carriages, mounts, positioners, medical instruments, imaging devices, position sensing devices, and/or the like. Any functionality described herein can be implemented by the control circuitry 211 of the robotic system 10 and/or the control circuitry 251 of the control system 50. Therefore, any reference herein to control circuitry may refer to circuitry embodied in a robotic system, a control system/tower, or any other component of a medical system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211, 251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

The control system 50 can further include various input/out (I/O) components 258 configured to assist the physician or others in performing a medical procedure. For example, the I/O components 258 can be configured to allow for user input to control/navigate an endoscope, sheath, and/or other instrument within a patient 1. The control system 50 can include one or more display devices 56 to provide various information regarding a procedure. For example, the one or more display devices 56 can be used to present a virtual anatomical model of the target anatomy with a virtual representation of a medical instrument having the proper relative position and shape as determined using processes described herein. The I/O components 258 can include user input controls 59, which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, toggle (e.g., button) inputs, and/or interfaces/connectors therefore. Such input(s) can be used to generate commands for insertion and/or retraction of medical instrument(s) and articulation of an articulating portion of the medical instrument(s), and/or to generate a command to change drive mode of the medical instrument(s).

### Robotic Instrument Localizing Systems

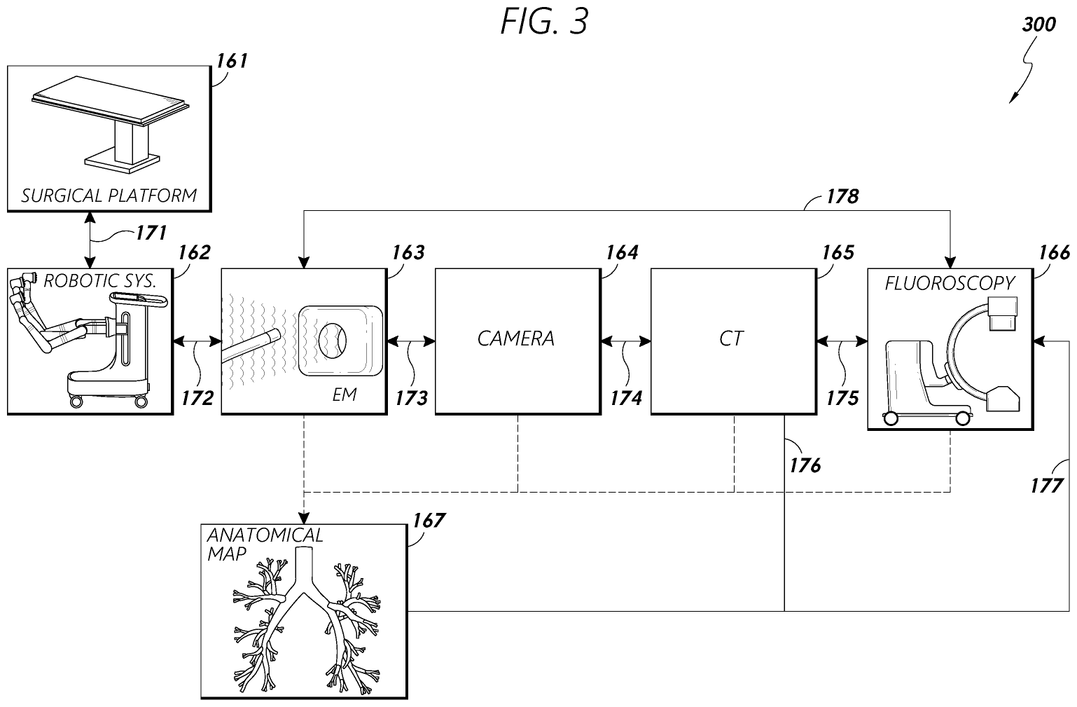

FIG. 3 is a block diagram illustrating a robotic instrument localization system 300 in accordance with one or more embodiments. In the context of the present disclosure, “localization” is used according to its broad and ordinary meaning and can refer to processes of determining the location and orientation/pose of an instrument within a given space or environment. The localization system 300 represents relationships and registrations between various positioning and imaging systems/modalities 161-167, which may be implemented to facilitate anatomical mapping and instrument navigation, positioning, and visualization for robotic procedures in accordance with aspect of the present disclosure. Each of the various systems 161-167 of the localization system 300 can be associated with a respective position coordinate frame and/or can provide unique data/information relating to instrument and/or anatomy locations, wherein registering the various coordinate frames to one another can allow for integration of the various systems to provide improved mapping, navigation, and instrument visualization. For example, registration of the various modalities to one another can allow for determined positions in one modality to be tracked and/or superimposed on/in a reference frame associated with another modality, thereby providing layers of positional information that can be combined to provide a robust localization system.

In various embodiments, the anatomical space in which an endoscope or other instrument may be localized (i.e., where position and/or shape of the instrument is determined/estimated) is a three-dimensional portion of a patient's tracheobronchial airways, vasculature, urinary tract, gastrointestinal tract, or any organ or space accessed via such lumens. Various localizing/imaging modalities may be implemented to provide images/representations of the anatomical space. Suitable imaging subsystems include, for example, X-ray, fluoroscopy, CT, PET, PET-CT, CT angiography, Cone-Beam CT, 3DRA, single-photon emission computed tomography (SPECT), MRI, Optical Coherence Tomography (OCT), and ultrasound. One or both of pre-procedural and intra-procedural images may be acquired in connection with a surgical procedure. Various subsystems as shown in the localization system 300 can provide information for generating a three-dimensional (3D) anatomical model/map (e.g., airway model) 167. Once the registered anatomical map 167 is available, the physician or automated control circuitry/system can use the map information during a procedure. For example, this may take the form of a visualization of a tracked instrument superimposed on the anatomical map with an estimated shape and position based on position sensors associated with the tracked medical instrument, as described in detail herein.

The localization system 300 includes a surgical bed or other patient platform or positioning/support structure 161. The position of the support structure 161 may be known based on data maintained relating to the position of the surgical bed within the surgical environment, or alternatively the position of the support structure 161 may be sensed or otherwise determined using one or more markers and an appropriate imaging/positioning modality.

The surgical system 300 may further include a robotic system 162, such as a robotic cart or other device or system including one or more robotic end effectors, as described above. Data relating to the position and/or state of positioners/actuator components of the robotic system 162 may be known or derived from robotic command data or other robotic data relative to a coordinate frame of the robotic system 162. Reference frame registration 171 between the support structure 161 and the robotic system 162 may be a relatively coarse registration based on robotic system/cart-set-up procedure, which may have any suitable or desirable scheme.

The surgical system 300 may further include an electromagnetic sensor system 163, which may include an electromagnetic field generator and one or more electromagnetic sensors, which may be associated with a portion of the instrument being tracked/controlled, such as along a length of an endoscope and/or other elongate member disposed in the working channel of the endoscope. In some implementations, the electromagnetic field generator may be mechanically coupled to either the support structure 161 or the robotic system 162, in which case registration 172 between such systems may be known and/or trivial. In some implementations, the field generator of the electromagnetic sensor system 163 can include one or more multiple-modality sensors integrated therewith, such as radiopaque fiducials, optical reflective markers, or the like. In some implementations, the registration 172 between the electromagnetic sensor system 163 and the robotic system 162 may be determined through forward kinematics and/or field generator mount transform information. For example, the field generator may be mounted to an end effector of the robotic system 162, such that the position of the field generator may be known relative to the robotic system positioning frame based on the known relationship between the position of the robotic end effector and the robotic system 162. The electromagnetic sensor system 163 may provide instrument pose and path information based on sensor readings associated with the instrument.

The system 300 may further include an optical camera system 164 including one or more cameras or other imaging devices, wherein such device(s) is/are configured to generate real-time images of patient anatomy within a visual field thereof during a surgical procedure. Registration 173 between the optical camera imaging system 164 and the electromagnetic sensor system 163 may be achieved through identification of features having electromagnetic sensors associated therewith, such as an endoscope tip, in images generated by the optical camera system 164. The registration 173 may further be based at least in part on hand-eye interaction of the surgeon when viewing real-time camera images while the EM-sensor-equipped endoscope is navigating in the patient anatomy.

The system 300 may further include a computed tomography (CT) imaging system 165 configured to generate CT images of the patient anatomy, which may be done preoperatively. Image processing may be implemented for registration 174 of the CT image data with the camera image data generated by the optical camera system 164. For example, common features or fiducials identified in both camera image data and CT image data may be identified to relate the CT image frame to the camera image frame in space. The CT imaging system 165 can advantageously be used to generate pre-operative imaging data for producing the anatomical map 167 and/or for path navigation planning.

The system 300 may further include a fluoroscopy imaging system 166 configured to generate real-time two-dimensional x-ray images of the surgical site. In some embodiments, the pre-procedural and/or intra-procedural images are acquired using a C-arm fluoroscope. The fluoroscopy imaging system 166 can be used with contrast to generate image data representing patient anatomy and instrumentation. The fluoroscopy imaging system 166 may be registered 175 to the CT imaging system 165 using any image processing technique suitable for such registration. The fluoroscopy imaging system 166 may further be registered 178 to the electromagnetic sensor system 163 through certain tool registration, such as by using one or more fiducial markers with known mechanical fixation/relationship to the field generator of the electromagnetic sensor system 163. For example, the mechanical structure of the C-arm instrumentation of the fluoroscopy imaging system 166 may have a known physical transform/relationship with respect to the mounting position of the electromagnetic field generator of the electromagnetic sensor system 163. Such known relationship can be used to register the fluoroscopy image space to the electromagnetic sensor image The connections 176, 177 represent registrations/relationships of the CT imaging system 165 and the fluoroscopy imaging system 166 to the anatomical map 167, respectively.

The position and/or shape of an instrument, such as an endoscope, may be determined using any one or more of the coordinate systems 161-166, which may facilitate generation of graphical interface data representing and image of the estimated position and shape of the instrument presented relative to the anatomical map 167, which may be displayed on a control system display device or other display device. The position and shape of an instrument can be determined based on shape estimation using EM sensors as described below or other shape estimation techniques.

### Instrument Position Sensor Integration for Shape Estimation

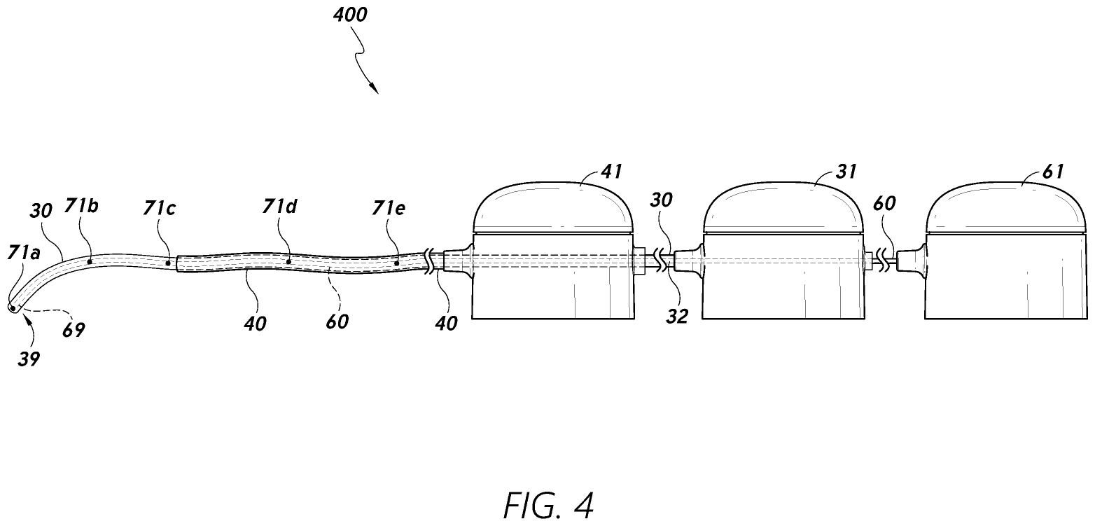

In accordance with aspects of the present disclosure, the shape of a medical instrument, such as a robotically controllable endoscope, may be determined using sensor devices, such as electromagnetic (EM) sensor devices, integrated with the medical instrument and/or other component/member associated therewith. FIG. 4 illustrates an assembly 400 of instruments that may be used in connection with a medical procedure, wherein any one or more of the various components of the assembly 400 may have a plurality of position sensors associated therewith for instrument shape estimation. As an example, the assembly 400 is shown as including an elongate medical instrument 30 (e.g., endoscope), an outer sheath 40, and a sensor probe 60. It should be understood that the assembly 400 may include or omit any of the illustrated components. Furthermore, example position sensors 71a-71e are shown (reference ‘71’ is used herein to refer to the position sensors 71a-71e individually and/or collectively for convenience and clarity) distributed along a length of the sensor probe 60. It should be understood that, as with any other embodiment disclosed herein, the sensors 71 can be integrated with the instrument 30, the probe 60, the sheath 40, or any other component of the described system/assembly.

The instrument 30 can include a base 31 (e.g., handle), which may be configured to be coupled to a robotic manipulator, as described above, to facilitate robotic instrument advancement/articulation. The instrument 30 may include one or more working channels 32 into which additional instruments/tools, such as the sensor probe 60, can be introduced. The instrument 30 can be configured to move/articulate in multiple degrees of freedom, such as: insertion, roll, and articulation in various directions, which can allow the instrument to assume various shapes, including possibly shapes with compound bends. In implementations in which an endoscope is manipulated within a controllable outer sheath, the system may provide even more degrees of freedom. The instrument 30 may be any type of elongate medical instrument, such as a bronchoscope, ureteroscope, laparoscope, or other type of endoscope, catheter, sheath, shaft, or the like.

The assembly 400 includes a plurality of position sensors 71a-71e, which may be used to estimate the shape of the instrument 30, or other component of the assembly 400, in accordance with any of the processes disclosed herein. For example, the sensors 71 can be disposed within, or otherwise integrated or associated with, the shaft of the instrument 30 along a length thereof. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, disposed at least partially within, connected to, integrated with, embedded at least partially within, or otherwise physically related to the second feature, element, component, device, or member, whether directly or indirectly.

In some exemplary embodiments, the sensors 71 may be electromagnetic (EM) sensors. However, it should be understood that any description of position sensors herein may be understood to refer to any suitable or desirable type of position sensor. The use of multiple sensors 71 distributed along a length of an elongate instrument/member can facilitate instrument shape estimation, as described herein, and represents an alternative to instruments including position sensors at only one lengthwise position/area thereof, such as one or more EM sensors at or near the tip of the instrument, which may not provide sufficient positional information from which to infer the shape of a length of the instrument. Not being aware of the shape of an instrument during a procedure can pose various challenges to using the instrument. For example, the lack of shape estimation information can make it difficult to determine how the instrument interacts with a patient's anatomy and/or to determine an optimal driving/navigation strategy.

The sensors 71 can be integrated in the body/shaft of the instrument 30, which may be advantageous as the positions of the sensors 71 can then correlate directly with corresponding portions of the instrument 30, such that the position and orientation of the sensors 71 can be analyzed to determine the shape of the instrument 30. However, integrating the sensor 71 in the shaft of the instrument 30 may introduce certain design complexities. For example, the integration of position sensors in the instrument 30 may increase the profile of the instrument to accommodate the physical volume of the sensors 71. Considering the space occupied by the working channel 32, the available volume of the shaft of the instrument 30 available for sensor integration may be limited in some cases. Therefore, it may be beneficial to implement the sensors 71 in one or more other components or devices of the assembly 400. In some implementations, the sensors 71 include a tip sensor 71a, as shown, which may be used to measure the position and orientation of the tip of the instrument 30. The relative positions of the sensors 71a-71f relative to one another and/or to a length of the instrument 30 and/or probe 60, as well as other physical specifications relating to the length of segment(s) of the instrument 30 and/or probe 60, may be predetermined and maintained in control circuitry of the relevant robotic system as parameters for use during real-time shape estimation according to processes disclosed herein. The physical material properties of the instrument 30 and/or probe 60 may also be predetermined and maintained for use in shape estimation. For example, stiffness parameters may be maintained that indicate bending stress response, which may be accounted for in shape estimation.

In some embodiments, the sensor 71 may be integrated in a separate elongate member, such as a sensor probe 60 configured to be inserted at least partially in/through the working channel 32 of the instrument 30. For example, the base 31 of the instrument 30 may provide a port for insertion of the sensor probe 60 into the working channel 32 of the instrument 30. The use of the sensor probe 60 within the working channel 32 of the instrument 30 for shape estimation, such that the probe 60 serves as a shape proxy for the instrument 30, can be suitable/feasible in certain procedural workflows. For example, the determination of the shape of the instrument 30 may be particularly useful during driving/advancement and articulation of the instrument 30 to a target anatomical site. During such procedural periods, the working channel 32 may often be unoccupied due to the lack of a need for working channel tools during such periods. Therefore, the working channel 32 may be available for use with the sensor probe 60, which may run along the length of the instrument 30 within the working channel 32 during instrument advancement/articulation. As the working channel 32 runs coaxially and/or parallel with the axis of the instrument 30, the probe 60 may assume/mimic the shape of the instrument 30 when disposed within the working channel 32. That is, the shape of the probe 60 can serve as a proxy/surrogate representing the shape of at least a portion of the instrument 30 when the probe 60 is disposed within the working channel 32 of the instrument 30. Therefore, by measuring sensor readings associated with the sensors 71 integrated/associated with the sensor probe 60 when the probe 60 is inserted within the instrument 30 as shown in FIG. 4, the shape of the instrument 30 can be estimated by determining/estimating the shape of the sensor probe 60 and imputing the shape of the sensor probe 60 to the instrument 30.

It may be desirable to implement the sensor probe 60 with a profile/diameter that is close to the inner diameter of the working channel 32 of the instrument 30, such that the sensor probe 60 fits relatively tightly within the working channel 32, while still providing enough clearance to allow for axial sliding of the probe 60 within the working channel 32. For example, if the sensor probe 60 has a diameter substantially less than the inner diameter of the working channel 32, the radial clearance between the probe and the inner walls of the working channel 32 may result in slack/play in the probe 60 within the working channel 32 that can result in the shape of the probe 60 varying undesirably from that of the working channel 32 and instrument 30. By relatively tightly fitting the probe 60 within the working channel 32, the shape of the probe 60 in the area of the sensors 71 may correlate sufficiently closely to the shape of the instrument 30 to allow for the sensor probe 60 to serve as a shape proxy for the instrument 30.

When inserted in the instrument 30, the tip 69 of the sensor probe 60 may advantageously be positioned close to and/or flush with the tip 39 of the instrument 30, such that measurement of the position of the sensor 71a, when integrated with the sensor probe 60, can be used to determine the position and orientation of the tip 39 of the instrument 30. In some implementations, the tip 69 of the sensor probe 60 is extended to project distally beyond the tip 39 of the instrument 30, or is positioned proximal of the tip 39 of the instrument 30 within the working channel 32. In some implementations, the position of the tip 69 of the sensor probe 60 may be selected to provide shape information relating to a selected length segment of the instrument 30. That is, it may be desirable to determine the shape of a particular length segment of the instrument 30 that is proximal of the tip 39. By inserting the sensor probe 60 to cover the relevant length segment of the working channel 32, the sensors of the sensor probe 60 may be used to determine the shape of the instrument 30 over the desired length segment thereof. In some implementations, the sensor probe 60 includes a base 61 associated with a proximal end of the sensor probe 60. As with the other bases 31, 41, the base 61 may be configured to be coupled to a robotic end-effector/manipulator.

The assembly 400 shows the instrument 30 and sensor probe 60 passing through an outer sheath 40, which may have a base 41 associated therewith. The base 41 may include one or more ports for insertion of the instrument 30 and/or sensor probe 60 that provides access to the inner lumen of the sheath 40. In some implementations, one or more of the sensors 71 may be integrated with the outer sheath 40. It should be understood that any description herein of axially/lengthwise distributed/offset position sensors may be understood to be optionally integrated with any elongate component of a robotic system, such as an endoscope or other elongate instrument, access sheath, probe or other working channel instrument/member, or the like. Furthermore, although five lengthwise distributed/offset sensors 71a-71e are shown, it should be understood that any number of sensors may be implemented in connection with embodiments disclosed herein.

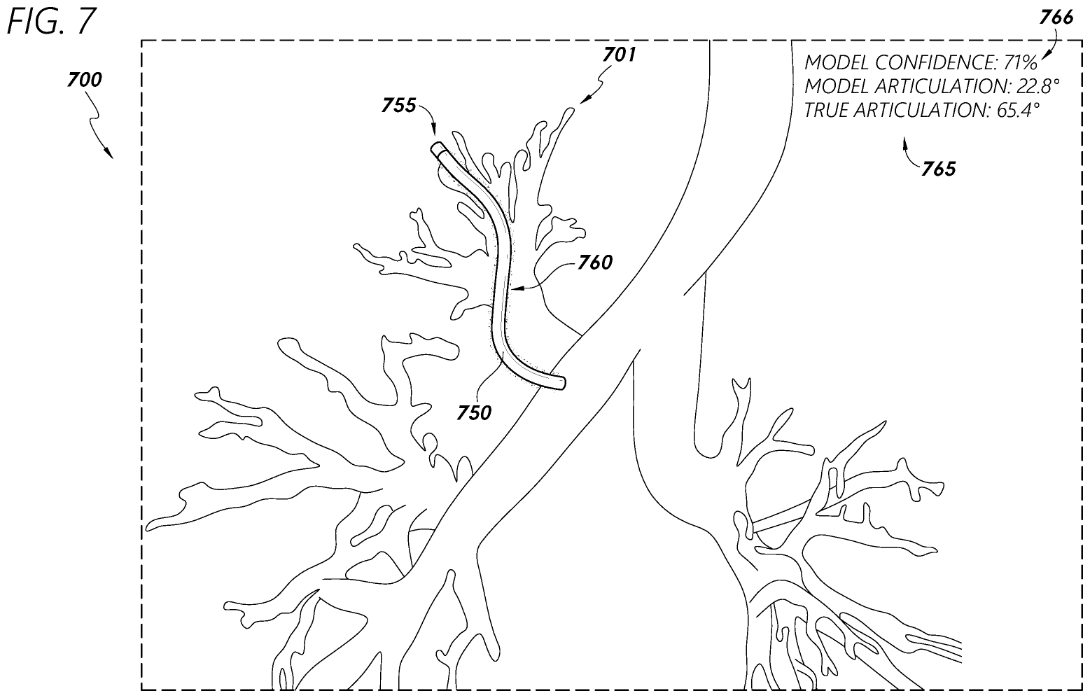

In implementations in which the sensors 71 are integrated with a working channel tool, such as the sensor probe 60, articulation of the instrument 30 can result in shortening of the instrument 30 to some degree due to mechanical compression. Accordingly, the positions of the sensors of the sensor probe 60 (e.g., working channel tool) relative to segment(s) of the instrument 30 may become altered slightly. Such condition can be accounted for using a confidence parameter that reflects a confidence level in the shape estimation, wherein the confidence level may decrease as the degree of articulation of the instrument 30 increases. The confidence parameter can be displayed in a user interface presenting a rendering of the instrument shape, as shown in FIG. 7 and described in further detail below. In some implementations, where the sensor probe 60 is robotically movable at the base 61, control circuitry of the robotic system may be configured to automatically move the sensor probe 60 in response to detected/determined displacement of the sensor(s) relative to the instrument 30 to compensate for such displacement. In some implementations, mechanical compression may be accounted for and compensated for within the relevant shape-estimation model. For example, the model may determine the location of the sensor(s) along the length of the instrument 30 as varying as a function of instrument articulation.

### Shape Estimation

The use of lengthwise-distributed position sensors as shown in FIG. 4 can allow for shape estimation in accordance with processes disclosed herein. Shape estimation processes disclosed herein can provide various benefits with respect to instrument navigation, including improvements in driving/navigation strategies, precision, and speed when navigating the instrument tip to a target compared to implementations lacking in shape information. For example, shape information as determined in connection with embodiments of the present disclosure can provide useful information relating to the true articulation of an instrument. Additional uses for instrument shape in robotic surgical systems include the following. With reference back to FIG. 4, for implementations where the outer sheath 40 does not include position sensor features, the associated navigation user interface may display the protrusion of the instrument tip 39 beyond the tip of the outer sheath 40. For example, such determination may be based on the distance between the instrument base 31 and outer sheath base 41. With information indicating the shape of the instrument 30, the location of the tip of the outer sheath 40 can be determined and/or visualized by placing a marker along the depicted shape of the instrument 30 at a distance equaling the distance between the instrument base 31 and the sheath base 41 from the instrument tip 39.

Furthermore, while some systems and processes involve the calibration of instruments prior to a procedure to provide improved device control, with instrument shape information, calibration can be additionally or alternatively performed intra-operatively. For example, control processes can be initialized with nominal control parameters that are updated in real-time. At least some of such updates may be performed during the registration between the position sensor system (e.g., EM) and the pre-operative anatomical model (e.g., CT). While some implementations are described herein in the context of EM sensors and sensor systems, it should be understood that utilization and determination of instrument shape information in accordance with aspects of the present disclosure can be based on other types of sensors and sensor systems.

In some implementations, an instrument shaft may be introduced to the internal patient anatomy after first passing through a patient introducer device/structure, which can include a rigid bend. Embodiments of the present disclosure can be implemented such that shape sensing is performed with respect to portions of an instrument that remain outside of the body during the relevant procedure and/or with respect of portions of an instrument between an instrument base/handle and a more-distal outer sheath base/handle. Sensed shape corresponding to such external instrument portion(s) can be used to determine a position of the introducer relative to the instrument.

In some implementations, control processes in surgical systems may utilize an estimated articulation angle of a tracked instrument, which can be inaccurate in some instances. Where articulation angle estimates are inaccurate, enforcing maximum articulation limits using such inaccurate estimates can undesirably limit further articulation of the instrument unnecessarily, which can prevent the instrument from reaching the commanded articulation. Through determination of shape estimates using lengthwise-distributed/offset position sensors in accordance with aspect of the present disclosure, accurate measurements of true instrument articulation can be determined where at least one of the distributed sensors is positioned at or near the base of articulation. In such implementations, articulation limits can be correctly enforced, thus avoiding the problem of premature articulation caps. Such shape estimation information can further allow for finer tip control, which can serve beneficial when positioned/articulated relatively close to a target. Instrument shape information can also enable closed-loop instrument control. Examples of closed-loop control applications that can be based at least in part on instrument shape information derived according to aspects of the present disclosure include lane-correction/center-line driving in anatomy through closed-loop control, and instrument auto-centering control to automatically orient the instrument tip towards a target. Instrument shape information can also be used for automated registration of pre-operative anatomical models (e.g., CT) to other positioning systems, such as robotic command or EM positioning frames.

Additional benefits that can be provided by shape estimation solutions as described herein include the ability to implement flexible anatomical (e.g., airway) models. For example, anatomical models (e.g., airway models in bronchoscopy) may be rigid in some implementations, in that they do not flex as the instrument passes through the anatomy, which can be unlike the natural behavior of the true anatomy. Furthermore, navigation processes in robotic surgical systems may force the displayed instrument tip to lie within the bounds of the anatomical model in some implementations. However, the forceful fitting of the instrument tip location to the rigid anatomical model can lead to inaccurate instrument tip localization. With knowledge of the instrument shape, as described herein, a flexible anatomical model can be implemented that can deform according to the determined instrument shape to accommodate the shape of the instrument, enabling more accurate depiction of the instruments position.

As another example, instrument shape determination as described herein can provide benefits with respect to instrument buckling detection. For example, with respect to some solutions, buckling detection implemented in surgical systems may only detect the presence of buckling of the tracked instrument, but not the precise location of the buckling. The lack of information relating to the location of instrument buckling can make it difficult for users to resolve buckling in an efficient way. This can delay procedures and/or cause the user to fail to reach the intended target. Shape determination processes as disclosed herein can provide indications of buckling based on the determined instrument shape. In addition, with a certain number and/or position of position sensors along the instrument length, the location of the buckling along the length of the endoscope can also advantageously be determined.

As yet another example of instrument navigation benefit provided by shape estimation processes disclosed herein, shape estimation can assist in navigation pathway selection. As an illustrative example, in robotic bronchoscopy, some airway selection solutions can utilize a probability map that determines a likelihood of an instrument tip being present in a given airway among a set of possible airways. However, it can be difficult to accurately predict the airway corresponding to the instrument tip position due to, for example, having data only from position sensor(s) disposed at the instrument tip. In some cases, this can lead to the visualized/determined tip location jumping back-and-forth between adjacent branch airways. Sensor redundancy as provided by the implementation of additional/multiple position sensors in an instrument or associated member/component as described herein, in addition information indicating true articulation, can provide improved probability mapping, resulting in higher confidence in the selected airway. Instrument shape information, when utilized for navigation pathway selection, can make airway selection substantially clearer, or even trivial. That is, the probability map can become more concentrated through evaluation against determined instrument shape.

Instrument shape determination and multi-sensor embodiments disclosed herein can further provide benefits relating to electromagnetic (EM) field distortion compensation. For example, in robotic endoscopy, an instrument tip may be localized using a tip-mounted EM sensor in some implementations. Many factors in the procedural setting and/or environment can affect the EM field, resulting in distorted EM data from the sensor. In some cases, it may be difficult to implement distortion compensation algorithms in surgical systems that can distinguish between EM field distortion and instrument motion caused by changes in the anatomy. Sensor redundancy as described herein can assist in distinguishing between EM field distortion and instrument motion. Moreover, determined instrument shape can be used to implement filters that better compensate for EM distortion.

Further, in certain robotic surgical procedures, such as robotic bronchoscopy, C-arm positioning can be a trial-and-error practice. When multiple attempts at attaining a suitable view of the target and the instrument, delays in a procedure can result. If a bronchoscope has only tip-mounted position sensor(s), a plane can be selected to fit to the position of the instrument, wherein the normal to the plane can be used to determine a desirable C-arm viewing angle. However, such plane fitting may require collection of a series of position sensor data over a period of time. Using historical position sensor data that is not representative of the current instrument position can lead to an inaccurate C-arm viewing angle. Shape estimation as described herein, which can involve generating a spline/curve that represents the tracked instrument's shape, can allow for the fitting of a plane to points along the spline/curve. The normal to said plane can be leveraged to determine an optimal/desirable viewing angle for the C-Arm that is optimal for real-time instrument shape/position.

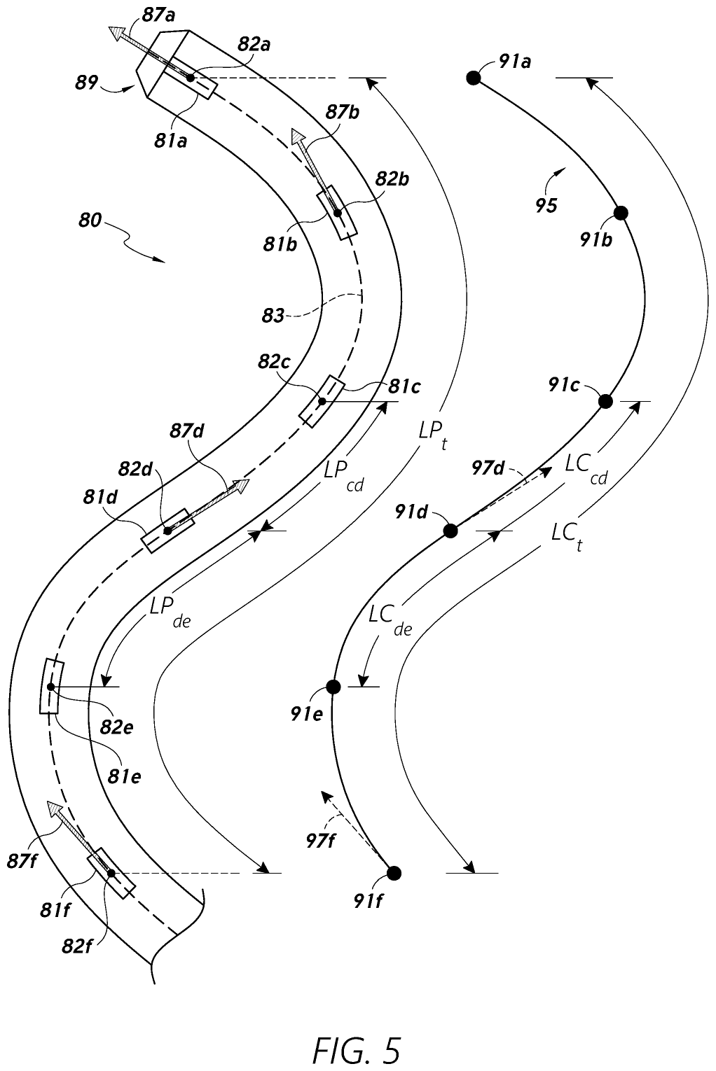

FIG. 5 illustrates an elongate instrument 80, which may be an endoscope (e.g., bronchoscope) in some implementations, or any other flexible instrument/member (e.g., sensor probe). The instrument 80 has associated therewith a plurality of position sensors 81a-81f (reference ‘81’ is used herein to refer to the position sensors 81a-81f individually and/or collectively for convenience and clarity), which may be used/measured to implement shape estimation in accordance with aspects of the present disclosure. The sensors 81 can advantageously be electromagnetic (EM) sensors, or any other type of position sensors. The sensors 81 can be positioned at various lengthwise positions/offsets along the length, or a length segment LPₜ, of the instrument 80, such that the shape of the instrument 80 can be determined based on position and orientation measurements from the multiple sensors 81. As the sensors 81 may not cover all positions along the length segment LPₜ of the instrument 80, determination of the shape of the instrument 80 based on sensor data from the sensors 81 may be considered estimation of shape. The sensors 81 may be integrated with the shaft of the instrument 80, or with a sensor probe that is disposed within a working channel of the instrument 80. In some implementations, the plurality of sensors 81 can be attached to or embedded within a jacket or sheath of the instrument 80. In the example of FIG. 5, the sensors 81 are represented as rectangles for simplification and illustration, though it should be understood that the sensors 81 can have any suitable shapes, dimensions, and/or configurations.

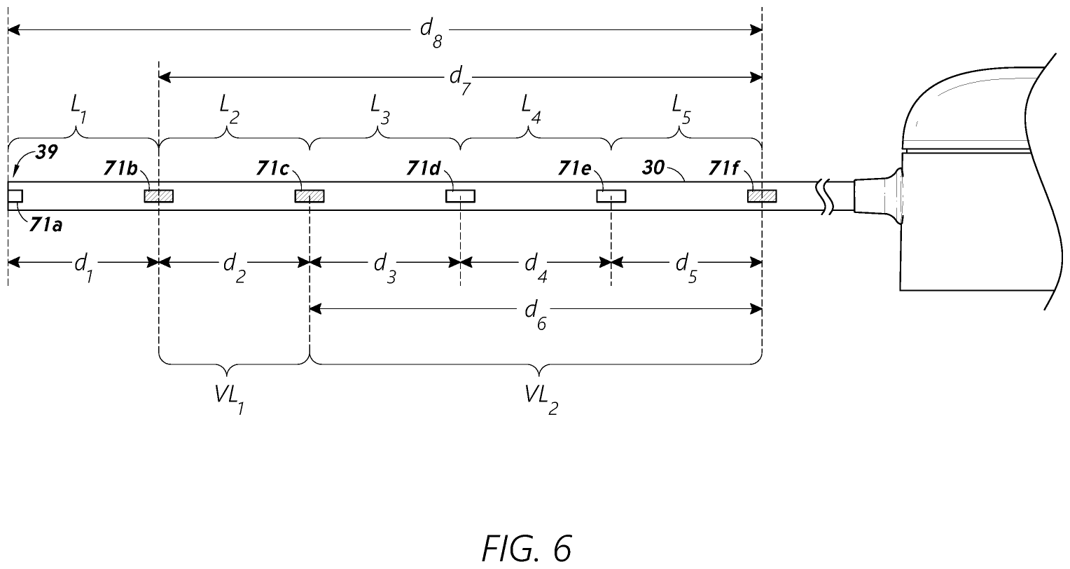

As an example, the instrument 80 is shown as including a first sensor 81a positioned at a location 82a at or near the tip 89 of the instrument 80, as well as multiple additional sensors 81b, 81c, 81d, 81e, 81f positioned proximal of the tip 89 at respective longitudinally-offset positions 82b, 82c, 82d, 82e, 82f along the shaft of the instrument 80. Six sensors 81 are shown for illustrative purposes, and any appropriate number of sensors can be implemented, covering any desirable length segment of an instrument, in connection with the embodiments of the present disclosure. The length segments of the instrument 80 between sensor pairs of the sensors 81 area referred to herein as “links.” Details relating to the configuration and dimensions of such links, including the implementation of “virtual” links, are described in greater detail below in connection with FIG. 6. In accordance with aspects of the present disclosure, the shape 83 of the instrument 80 in the length segment LPₜ can be estimated by generating a curve 95 that passes through a set of the center points 82a-82f at corresponding curve points 91a-91f and matches the respective headings (e.g., headings 87a, 87b, 87d, 87f) of the sensors at the points 82a-82f. The shape of the curve 95 can be used as an estimate of the shape of the instrument 80.

### Shape-Fitting Curve Generation Using Multiple Sensors

With further reference to FIG. 5, the shape of the instrument 80 can be estimated based on sensor measurements of two or more of the plurality of longitudinally-offset sensors 81 based at least in part on the know relative physical positions of the sensors 81 with respect to the length of the instrument 80. Such shape estimation can involve determining the curve 95 as a best-fit curve that matches the shape 83 of the instrument 80 as closely as possible. Matching the curve 95 to the shape 83 of the instrument 80 can involve determining a curve that passes through two or more of the sensors 81 and matches the orientations of the sensors at their respective positions. “Curves” described herein may be any type of continuous line or path, such as a spline or other piecewise defined curve. In some implementations, the curve 95 may be a third-degree spline defined by piecewise cubic polynomials. Types of curves that may be used include Hermite splines, Bezier curves, and the like. Using Hermite splines can provide desirable scalability to account for added sensors. For example, Hermite splines can generally be implemented in a modular fashion that can be beneficial for constructing curves that are a combination of a desirable number of sensor-to-sensor curve link segments, as described in detail below. In some examples, second-degree splines can be used for shape estimation. For instance, a constant curvature model can be used.

Shape-fitting curve determination can involve determining, for each of a plurality of lengthwise-offset sensors associated with an instrument, a position vector ‘X,’ including x, y, and z coordinate position values, and a quaternion ‘Q,’ including a set of scalar/real and/or vector/imaginary component values [q_{w}, qₓ, q_{y}, q_{z}] representative of sensor orientation. The position vectors for the plurality of sensors may be referenced as X₁, X₂, X₃, . . . Xₙ, wherein Xᵢ is the position vector of the i-th sensor. The orientation vectors for the plurality of sensors may be referenced as Q₁, Q₂, Q₃, . . . Qₙ, wherein Qᵢ is the quaternion of the i-th sensor. Heading direction of the individual sensors can be derived from the associated quaternion Q. The shape-fitting curve can be calculated using a model that generates a curve that passes through X coordinates of all or a subset of the sensors and has derivatives/slopes that correspond to the respective quaternions Q at the respective sensor positions. For example, with respect to FIG. 5, the shape-fitting curve 95 may be generated by determining a curve that passes through each of the sensor positions 82a-82f (or a subset thereof), wherein the curve 95 has derivative values (e.g., 97d, 97f) at each of the curve sensor positions 91a, 91b, 91c, 91d, 91e, 91f (which correspond to the physical positions 82a-82f of the sensors 81a-81f) that are based on the respective quaternion Q values. That is, the slope of the curve 95 at each sensor position 91a-91f is given by the corresponding quaternion Q associated with the given sensor. For illustration purposes, FIG. 5 shows derivative trajectories 97d, 97f of the curve 95, which match the quaternion Q orientations/headings 87d, 87f of corresponding sensors 81d, 81f.

The derivatives/slopes of the curve 95 at the sensor positions 91a-91f can be determined using an optimization process that involves a cost minimization function. In connection with the curve generation process, each of the sensors 81 can be measured to provide respective position vectors X and quaternions Q. Such X and Q determination may be implemented as a real-time process, such as during a medical procedure. The output of the process can comprise a curve (e.g., third-degree Hermite spline)f(x), wherein ‘x’ represents a distance/position along a length of the segment LPₜ of the instrument 80 that is represented by the curve 95. The curve can comprise a piecewise polynomial curve composed of individual sensor-to-sensor link segments defined through interpolation based on the position X and orientation/derivative Q data. Specifically, the output may be the solution to an optimization function for determining a derivative (df/dx) for each sensor position i (1, 2, 3, . . . n; n=6 in the example of FIG. 5) along a length of the instrument, wherein the optimization function implemented may be similar to equation (1) below in one or more respects:

$${Min}_{{( df / dx )}_{1} , {( df / dx )}_{2} , …} {w}_{1} ‖ {( link lengths )}_{f} - {( link lengths )}_{true} ‖ + {w}_{2} \vert {( total length )}_{f} - {( total length )}_{true} \vert \qquad ( 1 )$$

Constraints in the optimization function can be enforced by forcing df/dx to equal k*h, where ‘k’ is a positive scalar and ‘h’ is a heading vector. As a result, the optimization function can be reduced to a determination of the various scalars, k, at each sensor position X. The optimization function may be implemented such that there is a tolerable error bound about each of the constraints.

In equation (1) above, the term ∥(link lengths)_{f}−(link lengths)ₜᵣᵤₑ∥ represents the difference between the sensor-to-sensor link lengths LC between sensors represented by the curve/spline generated using a given derivative value and the actual known physical link lengths LP of the instrument 80, whereas the term |(total length)_{f}−(total length)ₜᵣᵤₑ| represents the difference between the overall curve/spline length LCₜ produced by the given derivative value and the known actual physical length LPₜ between the boundaries of the instrument represented by the curve 95. Variables w₁ and w₂ are weights that may be used in the calculation and tuned to produce the desired curve generation result, wherein either of the weights w₁, w₂ may be set to a value of zero. Therefore, in equation (1), the derivatives for each sensor position 91a-91f are determined as the derivative that produces the closest match (i.e., the lowest cost of the cost function) between the curve/spline link lengths LCₓₓ and the overall curve/spline LC length produced by the derivative value compared to the known actual physical link lengths LPₓₓ and known physical length LPₜ between the boundaries of the instrument represented by the curve. In some implementations, the various link lengths used in equation (1) can be determined using fluoroscopy and/or camera-based marker systems. In equation (1), the terms (link lengths)_{f} and (link lengths)ₜᵣᵤₑ may be vectors or arrays having a size that is equal to the number of sensor links. The term ∥(link lengths)_{f}−(link lengths)ₜᵣᵤₑ∥ represents the norm of the difference between corresponding link lengths. This norm is scaled by the weight w₁ in equation (1), which results in all link length differences are weighted equally according to the representation of equation (1) above. However, it should be understood that the weights associated with the link length differences can be varied. For example, the links closer to the tip of the instrument could be weighted higher. Equation (2) below is a representation of how such varied weighting could be implemented:

$$\sqrt{{l}_{diff}^{T} * W * {l}_{diff}} \qquad ( 2 )$$

In equation (2), l_{diff} can be equal to (link length)_{f}−(link length)ₜᵣᵤₑ; and W can be a diagonal weighting matrix with the same or varying weights.

As described above, the shape estimation curve 95 can be generated based at least in part on comparisons between computed curve link length segments and predetermined physical instrument link length segments. For example, in one use case, the shape estimation model can determine the derivative/trajectory 97d corresponding to the point 91d on the curve 95 associated with the sensor 81d by comparing generated curve segment lengths LC_{cd} and/or LC_{de}, which are adjacent to the point 91d on either side of the point 91d, to actual physical link lengths LP_{cd} and/or LP_{de}. That is, in the calculation of equation (1), (link lengths)_{f} may correspond to one or both of the lengths LC_{cd}, LC_{de}, whereas (link lengths)ₜᵣᵤₑ, may correspond to one or both of the actual known physical lengths LP_{cd}, LP_{de}. Further, the term (total length)_{f} may correspond to the entire length LCₜ spanned by the curve 95 between the sensor position points 91a, 91f, whereas the term (total length)ₜᵣᵤₑ may correspond to the actual physical length LPₜ of the instrument 80 spanning between the outermost sensors 81a, 81f. The various derivatives/slopes of the curve 95 can be selected/calculated derivatives/slopes that minimize the differences between such curve link lengths and known physical link lengths.

As described in detail above, the curve 95 is determined as a curve that passes through the sensors 81, wherein the sensors 81 may be integrated with the shaft of an instrument, such as an endoscope, or alternatively as a sensor probe configured to be disposed in a working channel of an instrument, as described in detail above in connection with FIG. 4. With reference back to FIG. 4, the sensor probe 60 may be robotically controllable in some implementations through mechanical connection with a robotic end effector, which may couple to a base 61 of the sensor probe. In some implementations, the various sensor readings of the probe 60 for shape estimation may be taken from a plurality of sensors 71 positioned along a length of the probe 60. However, in some implementations, sensor probe sensor readings at different lengthwise positions on the instrument shaft may be measured using fewer sensors than the number of sensor readings by sliding the sensor probe through the instrument working channel and taking sensor readings at different positions of the sensor(s). For example, with the sensor probe 60 mounted on an actuated base 61, the sensor probe 60 can be retracted and/or inserted one or more times along the length of the instrument 30 intra-operatively, such as while the instrument 30 is being driven by the user. Such process can produce finely sampled sensor positions X and/or quaternions Q along the length of the instrument 30. A spline can be fitted to these samples, wherein the resolution of the samples may allow for shape estimation without the need to solve an optimization problem according to equation (1) above. For example, in an embodiment where the instrument's mechanical design is known to be capable of only taking shapes represented by a n-th order polynomial, n+1 unique samples of the sliding sensor probe may be implemented to find the unique n-th order polynomial curve. Such processes can also be implemented using manually (i.e., non-robotic) controlled sensor probes. For example, a n-th order polynomial shape can be determined using n+1 sensors distributed along the length of the manually controlled instrument.

In some examples, a sensor probe can embody only a single sensor, which can be dynamically inserted in and/or out of the working channel, tracing the length of the shaft. A collection of points along the path traversed by the sensor can give a high-resolution representation of the shape of the instrument. Such dynamic insertion can be robotically controlled, wherein the system control circuitry can determine (e.g., from mechanical sensors) the distance the probe has been inserted at any given moment of sensor sampling. If the probe is robotically inserted such that the length/distance of insertion between samples is known, such length/distance can be considered to represent a virtual sensor link length, as described in further detail below.

In some implementations, shape estimation processes that comprise sliding a sensor through a working channel of a tracked instrument intra-operatively can provide sufficiently high spatial resolution along the length of the curve such that the shape of the instrument may be determined without regard to direction vectors (e.g., quaternions) at the sensor reading positions. Such implementations can therefore provide advantages with respect to computational burden. Sensor probes having as few as one sensor may be embodied in a disposable probe, which may provide certain cost benefits compared to more complicated devices.

Instrument shape estimation in accordance with systems and processes disclosed herein can be used to determine a buckling condition of an instrument. For example, an instrument can be considered buckled if the instrument's tip does not move (or respond) when the base of the instrument is inserted further into the patient anatomy. This results in the portions of the instrument being deformed at curvatures higher than the curvature of the patient anatomy (e.g., airway curvature). For example, control circuitry of robotic systems of the present disclosure can be configured to determine a buckling condition in response to a determination that the curvature of the shape estimate exceeds a known curvature threshold. The threshold can be set based on the pre-operative anatomical model, which may comprise a segmented anatomy in CT images. Shape confidence parameters, as described herein, may also be maintained and may indicate buckling. Generally, curves with varying and high curvatures cannot be approximated as well by lower-ordered splines, and therefore buckled instruments can result in a lower confidence curve fitting and/or shape estimation. In systems implementing a leader instrument within an outer sheath, buckling of an instrument can often be resolved at least partially by repositioning the outer sheath by retracting or inserting a base associated with the sheath. For example, buckling proximal to the tip can be at times resolved by inserting the outer sheath. Guidance and/or schemes relating to buckling resolution can be provided to the user through user interface engagement/presentation. Control circuitry can be configured to automatically resolve a buckling condition, once detected using shape estimate data, such as according to a robotic control script, which may operate based on the determined location of buckling and the control scheme/script.

### Physical and Virtual Link Lengths

As described above, shape estimation in accordance with aspects of the present disclosure can be based on comparisons of the lengths of sensor links/segments of an instrument to lengths of corresponding links/segments of a shape-fitting curve associated with the instrument. The link lengths can be used in the mathematical model described above. Although FIG. 5 shows links LP_{cd}, LP_{de} that span between adjacent sensors along the length of the instrument 80, it should be understood that instrument sensor links used for shape estimation comparisons can span between any desirable pair of sensors along the instrument length. As used herein, “physical sensor links” are links that span between adjacent sensors of an instrument, whereas “virtual sensor links” or “logical sensor links” are sensor links that span between non-adjacent sensors of an instrument but are nevertheless used as reference frames/lengths for comparing generated curve segments to actual known instrument segments, or links between adjacent sensors that are selected for comparison at the exclusion of other physical links. Sensor “links” described herein may refer to sensor-to-sensor length segments of an instrument or sensor-to-sensor length segments of a curve that is shape-matched to the instrument.

FIG. 6 shows an elongate instrument 30 having longitudinally-offset sensors 71a-71f, as described herein. The distance between adjacent pairs of the sensors 71a-71f can be uniform along the length d₈ (e.g., ‘total length’) of the instrument 30 that is associated with the sensors 71a-71f, or variable sensor-to-sensor distances may be implemented. For example, the distance d₂ between sensor 71b and 71c may be different from the distance d₄ between sensors 71d and 71e or the distance d₁ between the distal-most tip sensor 71a and the second sensor 71b. In some implementations, sensor-to-sensor distances between more-distal sensors (e.g., distances d₁ and/or d₂) may be shorter than more-proximal sensor-to-sensor distances (e.g., distances d₄ and/or d₅). Such sensor spacing may be advantageous as providing greater sensor sensitivity in the distal articulable segment(s) of the instrument 80 compared to proximal instrument portions, which may or may not be articulable. In examples in which variable sensor-to-sensor spacing is implemented, the various illustrated physical links L₁, L₂, L₄, and/or L₅ may likewise have variable lengths. In accordance with aspects of the present disclosure, shape-fitting curves can mirror the link configurations of the physical instrument, such as according to a sensor link scheme similar to that shown in FIG. 6. Although six sensors are shown in FIG. 6, it should be understood that any number of sensors may be implemented. In an example use case, only sensors 71b, 71c, and 71e are included, or more sensors are included in the instrument but only a subset of the sensors (e.g., sensors 71b, 71c, 71e) are considered for shape estimation. In some such implementations, the distances d₁ and d₂ may be equal (e.g., 30 mm), whereas the distance between sensor 71c and sensor 71e is twice the distance d₁/d₂.

In some implementations, shape estimation is performed based on virtual link length comparisons. For example, the subject instrument 30 may include a plurality of sensors, wherein one or more of the sensors may not be considered in generating a shape estimation curve. With respect to the diagram of FIG. 6, such an implementation may involve generating a curve that has a matched trajectory at only a subset of the sensors 71a-71f, such as sensors 71b, 71c, and 71f, as an example. In such an implementation, shape curve fitting may involve comparisons using virtual link lengths VL₁ and VL₂, wherein VL₂ spans between sensors 71c and 71f. The resulting shape estimation curve may therefore only have links corresponding to the virtual links VL₁, VL₂. Use of virtual links in shape estimation can help to reduce computational complexity. A virtual link may correspond to a physical link or correspond to multiple physical links, depending on the embodiment. With respect to FIG. 5, for example, any of the various referenced links LP, LC may be physical links, or alternatively may be virtual links of any desired configuration/re-configuration relative to the actual physical layout of sensors associated with the instrument 80.

The configuration of virtual links for shape estimation may be dynamically adjusted to increase shape estimation speed, reduce computational load, and/or to focus shape estimation on an instrument segment of interest. In certain embodiments, the virtual link boundaries may be variable over time. In some implementations, the virtual link boundaries may dynamically change as a function of a current articulation state of the instrument. For example, the virtual link lengths may be shortened or otherwise re-configured in response to increased articulation.

### Instrument Shape Visualization

FIG. 7 illustrates a user interface 700 for displaying a shape of an instrument and related information, in accordance with aspects of the present disclosure. The user interface 700 can provide a visualization of the shape of an instrument. The shape of the instrument may be determined using shape estimation processes disclosed herein, wherein the shape of the represented instrument 750 is determined using longitudinally-offset sensors integrated in the instrument or in a working channel probe as described in detail herein.

In the example of FIG. 7, a rendering of an instrument 750, such as a bronchoscope, is shown in the user interface 700. The instrument representation 750 can be overlaid, superimposed, or otherwise represented in a determined position and orientation relative to an anatomical map/model 701, which may be an airway model of a patient, for example. The visualization can advantageously show the tip 755 of the instrument 750, which may be helpful to the user when navigating the instrument through the anatomy.

The user interface 700 may display certain information 765 to the user, such as information associated with a procedure, the anatomical model 701, the shape of the presented instrument, etc. In some embodiments, the rendering of the shaped instrument 750 can be generated to follow a shape-fitting curve/spline fitted to a plurality of position sensors (e.g., EM sensors) determined in accordance with solutions presented herein. The information 765 can advantageously include a confidence measure 766 reflecting a level of confidence in the displayed instrument shape. The confidence measure 766 can be a function of the value of the optimized cost function. In some implementations, the information 765 further includes a measure of articulation expected based on robotic commands and/or a robotic kinematic model, as well as a measure of true articulation as determined using the position sensors associated with the instrument.

The visualization of the shaped instrument 750 can be generated and displayed in real-time. In some cases, as articulation of the instrument 750 increases, the confidence measure 766 associated with the shape of the instrument 750 may decrease. The confidence measure may be visually displayed as a halo/outline 760 around the instrument 750, or other non-text visual indicator. For example, the intensity (e.g., width, brightness) of the outline 760 may increase as the confidence measure decreases and decrease as the confidence measure increases, or vice versa. In this way, the outline 760 can provide a warning when the confidence measure decreases and/or a confirmation when the confidence measure increases. In certain embodiments, the color of the outline 760 may change as the confidence measure changes. In some implementations, a marker may be displayed along the shape 760 of the instrument representing the position of the outer sheath tip. The distance between the added marker and the displayed instrument tip may be, for example, the distance between the instrument base 31 and sheath base 41.

### Shape Estimation Processes

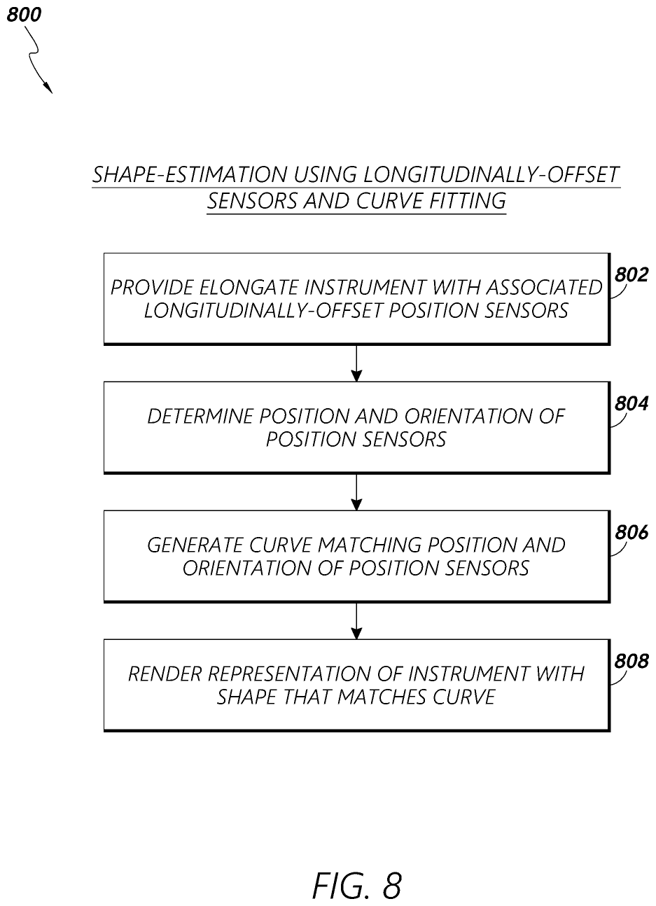

FIG. 8 is a flow diagram illustrating a process 800 for performing shape estimation using longitudinally-offset sensors associated with an instrument, such as an endoscope or other medical instrument, in accordance with aspects of the present disclosure. The process 800 may be implemented in connection with any of the embodiments disclosed herein. Furthermore, steps of the process may be performed by control circuitry of any of the robotic systems disclosed herein, at least in part.

At block 802, the process 800 involves providing an elongate instrument having associated therewith a plurality of longitudinally-offset position sensors. For example, the instrument may comprise an endoscope or similar shaft-type medical instrument. The plurality of sensors may be associated with the instrument in any suitable or desirable manner. For example, the sensors may be integrated with and/or disposed on a shaft structure of the instrument. In some implementations, the sensors may be associated with the instrument in that the sensors may be integrated with an elongate member of a probe device/tool configured to be disposed within a working channel of the instrument that runs parallel with an axis of the instrument. In such embodiments, the positioning of the elongate probe within the working channel of the instrument can result in the probe conforming to the shape of the instrument, such that shape determination with respect to the probe can be imputed to the instrument to provide a shape estimation.

At block 804, the process 800 involves determining position and orientation states of the plurality of sensors associated with the instrument. For example, the sensors may be measured/read in a manner as to provide positional information in the form of x-y-z coordinate frame position vectors, as well as slope/orientation information associated with each of the sensors, such as in the form of quaternion data structures, as described in detail herein. In some implementations, the sensors are electromagnetic sensors configured to have electrical currents induced therein in response to electromagnetic field exposure, wherein such currents are indicative of the position of the sensors relative to the electromagnetic field source.

At block 806, the process 800 involves generating a curve, such as a spline, which intersects each of the determined sensor positions and has slope features correspondent to each of the sensor positions that match the determined orientations of the sensors in such areas.

At block 808, the process 800 involves rendering a representation of the instrument on a graphical interface that shows at least a portion of the instrument having a shape that matches the shape of the generated curve. For example, the rendering of the instrument may be displayed on, and in relative position and orientation with, a virtual model of an anatomical area in which the instrument is presently disposed. By rendering the representation of the instrument in its estimated curved shape, the process 800 may advantageously provide useful information to the user to assist in guiding navigation, articulation, and/or the like.

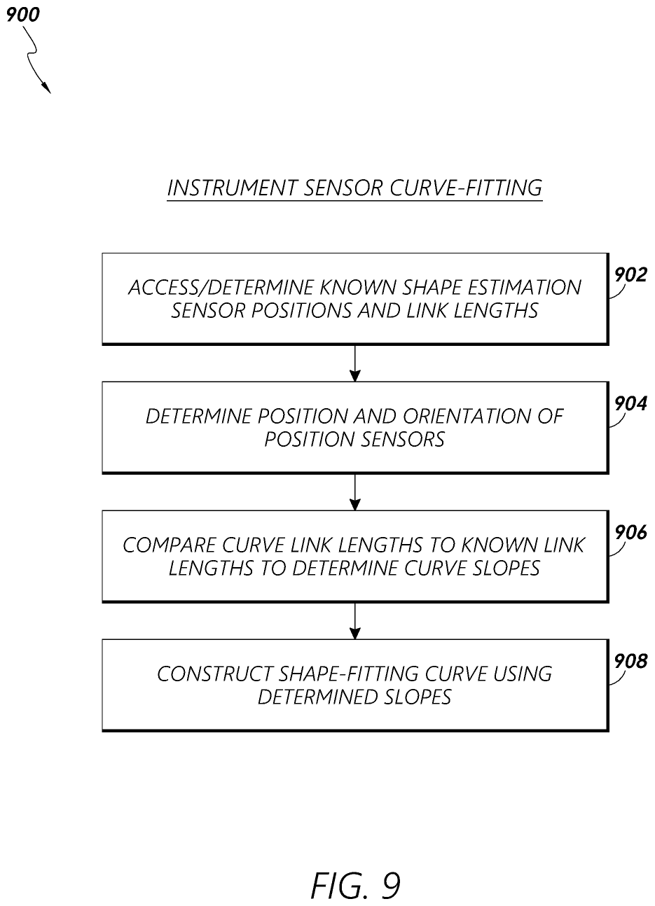

FIG. 9 is a flow diagram illustrating a process 900 for fitting a curve to position sensors distributed about a length of a target instrument, such as an endoscope or other medical instrument, in accordance with aspects of the present disclosure. The process 900 may be implemented in connection with instrument shape estimation processes as described herein. Furthermore, steps of the process may be performed by control circuitry of any of the robotic systems disclosed herein, at least in part.

At block 902, the process 900 involves accessing and/or otherwise determining known positions and link lengths associated with shape estimation sensors distributed along a length of the instrument. For example, the sensor positions and link lengths may be defined with respect to positions of a plurality of longitudinally-offset position sensors relative to the length dimension of the instrument. The link lengths may be physical or virtual link lengths, as described herein, and may represent length segments of the instrument that span between position sensors of interest with respect to instrument shape estimation.

At block 904, the process 900 involves determining position and orientation states associated with the plurality of sensors associated with the instrument. For example, the sensors may be measured/read in a manner as to provide positional information in the form of x-y-z coordinate frame position vectors, as well as slope/orientation information associated with each of the sensors, such as in the form of quaternion data structures, as described in detail herein. In some implementations, the sensors are electromagnetic sensors.

At block 906, the process 900 may involve determining curve slope features for a curve/spline fitted to the array of sensors positioned along the length of the instrument. Determination of the curve slope features (e.g., derivative values) may involve generating curve segments corresponding to the link lengths of the instrument based on the orientation information and comparing the generated curve segment lengths to the actual known physical lengths of the instrument links, and further selecting slope values that minimize the discrepancy between the curve link lengths and the true instrument link lengths. The link lengths of the instrument may be selected/determined dynamically, such as by configuring virtual link lengths as described herein, or may be set prior to implementation of the process 900.

At block 908, the process 900 involves constructing a shape-fitting curve using the determined slope features/values. For example, curve segments having the determined slope features may be compiled/constructed together in a piecewise manner to form a continuous polynomial curve, which may have the characteristics of a spline (e.g., third-degree Hermite spline), or other type of curve.

Shape estimation in accordance with any of the processes disclosed herein can provide various uses. For example, given a shape estimate of an instrument tracked relative to an anatomical model (e.g., airway model), the contours of model (e.g., airway contours) and/or the mechanics of the instrument (e.g., endoscope) and/or the forces acting upon the instrument can be determined. Such forces can be visualized on a user interface to aid the user in lessening forces imposed on the anatomy. In addition, surgical instruments (e.g., endoscopes) often may need to be calibrated before use. Any such pre-procedural calibrations can be reduced in time or eliminated by utilizing live shape estimation during the procedure. Parameters that might otherwise need to be calibrated prior to the procedure may advantageously be tunable online, mid procedure. Furthermore, with available real-time shape estimation information, finer control of the target instrument can advantageously be achievable by utilizing the shape estimate as feedback in a closed-loop control process. For example, given a target pose, control circuitry can be designed to automatically drive the instrument. Furthermore, as surgical instruments can degrade with use mid-procedure, control circuitry can be designed to detect and account for such changes in dynamics during a procedure.

### Additional Implementation Description and Terminology

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A system, comprising:
an elongate instrument having a plurality of position sensors associated therewith, the plurality of position sensors being distributed along a length of the elongate instrument; and
control circuitry configured to:
determine positions and orientations of each of the plurality of position sensors;
determine a curve that passes through each of the respective positions of the plurality of position sensors and the respective orientations; and
estimate a shape of the elongate instrument based on the curve.

2. The system of claim 1, wherein the plurality of position sensors are integrated with a shaft of the elongate instrument.

3. The system of claim 1, wherein the plurality of position sensors are integrated with an elongate member disposed at least partially within a working channel of the elongate instrument.

4. The system of claim 1, wherein the control circuitry is further configured to render a representation of at least a portion of the elongate instrument on a graphical interface, wherein the representation of the at least a portion of the elongate instrument has a shape that is based on the estimated shape of the elongate instrument.

5. The system of claim 1, wherein the curve is a third-degree Hermite spline.

6. The system of claim 1, wherein the control circuitry is further configured to determine sensor link lengths associated with the plurality of position sensors.

7. The system of claim 6, wherein the curve is based at least in part on the sensor link lengths.

8. The system of claim 7, wherein at least one of the sensor link lengths spans between non-adjacent sensors of the plurality of position sensors.

9. The system of claim 7, wherein the control circuitry is further configured to re-configure the sensor link lengths during operation of the elongate instrument.

10. A system, comprising:
control circuitry configured to communicatively couple to an elongate instrument having a plurality of position sensors distributed along a length thereof;
wherein the control circuitry is configured to:
determine relative positions of the plurality of position sensors with respect to the length of the elongate instrument;
determine sensor link lengths associated with the plurality of position sensors, the sensor link lengths spanning lengths of the elongate instrument between pairs of the plurality of position sensors; and
generate a curve that represents a shape of the elongate instrument based on the sensor link lengths.

11. The system of claim 10, wherein the determining the relative positions of the plurality of position sensors and the determining the sensor link lengths involves accessing pre-determined parameters associated with the elongate instrument.

12. The system of claim 10, wherein the control circuitry is further configured to:
determine current positions of the plurality of position sensors relative to a coordinate system; and
determine current orientations of the plurality of position sensors relative to the coordinate system;
wherein the curve is based on the current positions and the current orientations.

13. The system of claim 12, wherein the control circuitry is further configured to:
generate one or more curve segments based on one or more of the current orientations; and
compare lengths of the one or more curve segments to respective ones of the sensor link lengths;
wherein the curve is based on the comparing.

14. The system of claim 13, wherein the control circuitry is further configured to:
determine a total length of the curve;
compare the total length of the curve to a predetermined length of the elongate instrument; and
determine that the curve is a best-fit curve based on the comparing the lengths of the one or more curve segments and the comparing the total length of the curve.

15. The system of claim 10, wherein the elongate instrument is an elongate probe configured to be disposed within a working channel of an endoscope.

16. The system of claim 10, wherein the determining the sensor link lengths is performed in real-time in connection with the determining the curve.

17. A method comprising:
determining positions of a plurality of position sensors distributed along a length of an elongate instrument relative to a coordinate system;
determining orientations of the plurality of position sensors relative to the coordinate system; and
generating a curve that passes through each of the positions of the plurality of position sensors at the respective orientations of the plurality of position sensors.

18. The method of claim 17, further comprising inserting an elongate probe within a working channel of the elongate instrument, the plurality of position sensors being integrated with the elongate probe.

19. The method of claim 17, further comprising generating graphical interface data representing:
an anatomical model; and
a representation of a segment of the elongate instrument conforming to a shape that matches the curve within the anatomical model.

20. The method of claim 17, further comprising determining sensor link segments associated with the plurality of position sensors, wherein the curve is based on lengths of the sensor link segments.
